(19) United States
(12) Patent Application Publication
(10) Pub. No.: US 2015/0316529 A1
(43) Pub. Date: Nov. 5, 2015

(54) SYSTEM AND METHOD FOR REAL-TIME ANALYSIS OF MOLECULAR SEQUENCES USING NANOCHANNELS

(71) Applicants: Jung Bum CHOI, Cheongju-si (KR); Jong Jin LEE, Cheongju-si (KR)

(72) Inventors: Jung Bum CHOI, Cheongju-si (KR); Jong Jin LEE, Cheongju-si (KR)

(73) Assignee: Chung National University Industry-Academic Cooperation Foundation, Cheongju-si, Chungcheonbuk-do (KR)

(21) Appl. No.: 14/387,190

(22) PCT Filed: Apr. 25, 2012
(86) PCT No.: PCT/KR12/03154

§ 371 Date: Sep. 22, 2014

(30) Foreign Application Priority Data
Apr. 6, 2012 (KR) 10-2012-0035945

(51) Int. Cl. G01N 33/487; C12Q 1/68; B01L 3/00; G01N 27/447; G01N 27/453

(52) U.S. Cl. CPC G01N 33/48721; G01N 27/44791; G01N 27/44765; G01N 27/453; B01L 3/50273; B01L 3/502746; C12Q 1/6869; B01L 2200/143; B01L 2300/0896; B01L 2400/0421; B01L 2400/0487; B01L 2400/082

(57) ABSTRACT

The present invention relates to a system for analyzing molecular sequences, which is capable of decoding unit molecules constituting various biopolymers on a real-time basis using nanochannels. A control electrode serves to control the unit molecules passing along the channel such that the velocity of movement, arrangement, and directivity of the unit molecules can be rendered uniform. Particularly, at least four probe electrodes are separately formed in the case of decoding ss-DNA base molecules. Each probe electrode is coated with four different types of DNA base molecules to maximize detection efficiency through the interaction with complementary base molecules moving along the inside of the channel.

### CROSS-REFERENCE TO RELATED APPLICATIONS

This is a national stage application of PCT/KR2012/003154, filed on Apr. 25, 2012, claiming priority to KR10-2012-0035945, filed on Apr. 6, 2012

### STATEMENT REGARDING FEDERALLY SPONSORED RESEARCH

Not applicable.

### BACKGROUND OF THE INVENTION

The present invention relates to a system for analyzing the sequence of molecules using a nanochannel, and more particularly to a system and method for analyzing the sequence of molecules using a nanochannel, in which a control electrode and probe electrodes are disposed on or around a nanochannel, and the types of individual molecules in a biological polymer passing through the nanochannel are read in real time by sensing either the change in charge distribution induced by the electrical dipoles of different individual molecules or the change in current caused by the orbital energy of the individual molecules while uniformly controlling the moving speed, arrangement and direction of the individual molecules of the biological polymer passing through the nanochannel.

Reading the sequence of individual molecules of biological polymers (for example, the amino acid sequence of molecules such as polypeptides or proteins, or the sequence of DNA base molecules) is very important in the understanding of biological information processing mechanisms. As a representative example, DNA is the whole of genetic information and consists of nucleotide units. A protein is synthesized based on the sequence of nucleotides encoded in deoxyribonucleic acid (central dogma), and if DNA has a mutated nucleotide sequence different from the original nucleotide sequence, a protein cannot be synthesized or a completely different protein can be synthesized to cause serious physiological problems. Thus, examining whether DNA has a correct nucleotide sequence is very important in terms of the prevention and treatment of diseases. As a result of the human genome project, the human genetic map has been constructed, and thus pathological diagnosis and treatment at the genetic level have been increasingly activated.

There are a total of four types of nucleotides, and each nucleotide consists of an identical pentose (deoxyribose), an identical phosphate group, and one of four types of bases: adenine (A), guanine (G), cytosine (C), and thymine (T). Herein, A and G are purines having a bicyclic structure, and C and T are pyrimidines having a monocyclic structure.

A variety of DNA sequencing methods have been developed, including early methods such as the Maxam-Gilbert sequencing method and the chain-termination method, and recent methods such as the dye-terminator sequencing method. However, such methods have disadvantages in that the number of bases that are analyzed per unit time is small and in that preliminary operations such as radioactive isotope substitution or dying are time-consuming. Moreover, such methods have disadvantages in that they are costly and discharge environmental pollutants such as radioactive waste after analysis. In addition, there is a limit to the length of DNA that can be analyzed, and there is difficulty in analyzing a number of DNAs at the same time. In view of such various problems occurring in the conventional molecule sequencing methods, the recent rapid development of nanotechnology will provide potential alternative technology for real-time sequencing of molecules in combination with biotechnology. This nanobiotechnology is currently in the research and development stage, but if it is realized in the future, it will be more simple and accurate and can significantly reduce the time required for molecular sequencing, compared to the conventional chemical methods as described above.

### BRIEF SUMMARY OF THE INVENTION

The present invention has been made in order to solve the above-described problems of the conventional system for analysis of individual molecules of biopolymers by the use of nanotechnology, and it is an object of the present invention to provide a system for real-time analysis of the sequence of molecules, which can reduce the excessive time required for preliminary operations, can fundamentally eliminate the discharge of environmental pollutants such as radioactive waste, and can accurately analyze the sequence of individual molecules at high speed.

A system for analyzing the sequence of molecules using nanochannels according to the present invention can be used to read the sequence of individual molecules of various biopolymers, for example, polypeptides, proteins or DNA. Specifically, the system for analyzing the sequence of molecules according to the present invention comprises: at least one nanochannel having a width and height that allows the individual molecules of a biopolymer (e.g., the amino acids of protein, or the base molecules of ss-DNA) to pass therethrough without twisting or folding; at least one control electrode disposed on any one side of the nanochannel across the nanochannel and configured to align the individual molecules, which are introduced into the nanochannel, in the same direction according to the electrical or chemical properties of the individual molecules; one or more probe electrodes, one end or side of which is disposed adjacent to one side of the nanochannel in a direction perpendicular to the lengthwise direction of the nanochannel and which are configured to sense either the change in charge distribution induced by the electric dipoles of different individual molecules passing through the nanochannel or the change in current caused by a difference in the orbital energy of the individual molecules; and a measurement element configured to measure the absolute or relative value of the change in charge distribution or current sensed by the probe electrodes.

Herein, the probe electrodes may be coated with complementary molecules capable of chemically bonding with the individual molecules of the biopolymer, respectively, in order to enhance their interaction with the individual molecules of the biopolymer passing through the nanochannel. For example, when the sequence of ss-DNA base molecules passing through the nanochannel is to read, at least four probe electrodes are formed separately, and are coated with four different DNA base molecules (T, G, A, and C), respectively, so that the DNA base molecules can form a chemical bond (T-A or C-G) with complementary base molecules that are introduced into the nanochannel, thereby maximizing the sensing efficiency of the probe electrodes.

The probe electrodes may be formed of a conductive or semi-conductive material including gold, silver, copper, platinum, palladium, titanium, nickel, cobalt, graphene, graphite, or carbon nanotubes.

The control electrode may be formed of a conductive material including gold, silver, copper, platinum, palladium, titanium, nickel or cobalt, and may be disposed on or under the nanochannel or under a substrate, so that it may be applied with a specific voltage, or earthed or floated.

Alternatively, the control electrode may be formed of a material, which includes graphene, graphite or carbon nanotubes and is capable of interaction with the individual molecules (e.g., π-π energy resonance with DNA nucleotide base molecules), and it may be disposed on or under the nanochannel or under a substrate, so that it may be applied with a specific voltage, earthed or floated.

Each of the probe electrode and the control electrode is composed of a single-layer electrode or a multilayer electrode, and at least a portion of the lower portion of the single-layer electrode or the upper or lower layer of the multilayer electrodes may be coated with a dielectric layer.

Meanwhile, the measurement element may be any one of a field-effect transistor (FET), an operational amplifier, a single-electron transistor (SET), a high-frequency single-electron transistor (RF-SET), a quantum point contact (QPC) and a high-frequency quantum point (RF-QPC).

The nanochannel may be open at one side, and at least one of the probe electrode and the control electrode may be disposed on the open side of the nanochannel.

In addition, at least one of the width and height of the nanochannel may decrease continuously or stepwise from the inlet toward the downstream side thereof so as to allow the individual molecules to pass therethrough without twisting or folding.

Also, at least a portion of the inner side of the nanochannel may be coated with a dielectric layer.

In an embodiment of the present invention, the measurement element may be electrically connected with the probe electrode through an extended gate, and may be at an atmosphere temperature lower than the atmosphere temperature of the nanochannel.

Further, the measurement element may be formed integrally on a substrate having the nanochannel formed thereon.

The system of the present invention may comprise one or more probe electrode pairs, each consisting of two opposite probe electrodes that are located at the two opposite sides of the nanochannels, respectively, and the probe electrode pairs may be connected to different measurement elements.

A method for analyzing the sequence of molecules using a nanochannel according to the present invention comprises the steps of: moving a biopolymer in a nanochannel by electrophoresis or a difference in pressure of a fluid; applying a voltage to a control electrode formed on or under the nanochannel or under a substrate having the nanochannel formed thereon, or connecting the control electrode to an earth, or floating the control electrode, to control the direction of individual molecules of the biopolymer (e.g., nucleotide bases included in ss-DNA); inducing a change in charge distribution of a probe electrode by the individual molecules; and transferring the change in charge distribution to a measurement element to read the type of individual molecules.

Alternatively, a method for analyzing the sequence of molecules using a nanochannel according to the present invention comprises the steps of moving a biopolymer in a nanochannel by electrophoresis or a difference in pressure of a fluid; applying a voltage to a control electrode formed on or under the nanochannel or under a substrate having the nanochannel formed thereon, or connecting the control electrode to an earth, or floating the control electrode, to control the direction of individual molecules of the biopolymer (e.g., nucleotide bases included in ss-DNA); tunneling the energy levels of the individual molecules through a probe electrode pair consisting of two opposite probe electrodes; and sensing a change in the tunneling currents by a measurement element connected to the probe electrode pair to read the type of individual molecules.

Alternatively, a method for analyzing the sequence of molecules using a nanochannel according to the present invention comprises the steps of moving a biopolymer in a nanochannel by electrophoresis or a difference in pressure of a fluid; applying a voltage to a control electrode formed on or under the nanochannel or under a substrate having the nanochannel formed thereon, or connecting the control electrode to an earth, or floating the control electrode, to control the direction of individual molecules of the biopolymer (e.g., nucleotide bases included in ss-DNA); interacting the individual molecules with a single-layer probe electrode or the lower-layer electrode of a multilayer probe electrode, formed on the nanochannel; and sensing either a change in current of the single-layer electrode or a change in current of the lower-layer electrode of the multilayer probe electrode, caused by a change in a voltage applied to the upper layer electrode, by a measurement element connected to the single-layer electrode or the lower layer electrode of the multilayer probe electrode, to read the types of individual molecules.

In a method that is applied to all the methods for analyzing the sequence of molecules using the nanochannel, a plurality of probe electrodes or probe electrode pairs having the same configuration may be formed per nanochannel, so that the sequence of individual molecules of a biopolymer (e.g., ss-DNA or polypeptide) that passed through the nanochannel can be independently read a plurality of times, thereby increasing the reliability of analysis while greatly reducing the time required for analysis. This is the most important key element in the implementation of the present invention, and it is to be understood that, as the number of the plurality of probe electrodes increases, the speed and reliability of analysis of the base sequence increase. However, all the probe electrodes should be disposed within the range of the nanochannel.

In a method that is applied to all the methods for analyzing the sequence of molecules using the nanochannel, the probe electrodes may be coated with complementary molecules capable of chemically bonding with the individual molecules, respectively, which pass through the nanochannel, in order to enhance their interaction with the individual molecules. For example, if the sequence of ss-DNA base molecules passing through the nanochannel is to read, at least four independent probe electrodes may be formed, and may be coated with four different base molecules (T, G, A, and C), respectively, so that they can form a chemical bond (T-A or C-G) with complementary base molecules moving along the channel, thereby maximizing the sensing efficiency.

According to the present invention, a control electrode is disposed on or under a nanochannel, so that the moving speed, arrangement and direction of the individual molecules of a biopolymer passing through the nanochannel are maintained uniformly by the control electrode. In addition, the change in charge distribution or current induced by the molecules passing through the nanochannel is sensed by one or more probe electrodes, and the type of each of molecules is analyzed in real time by a measurement element. Thus, the present invention has an advantage in that the sequence of individual molecules can be precisely analyzed at high speed without causing environmental pollution. Particularly, if ss-DNA base molecules are to read, at least four independent probe electrodes may be formed, and the probe electrodes may be coated with four different types of DNA base molecules, respectively, so that the probe electrodes can interact with complementary base molecules passing through the nanochannel, thereby maximizing the sensing efficiency and reliability.

### BRIEF DESCRIPTION OF THE SEVERAL VIEWS OF THE DRAWINGS

FIG. 1 is a perspective view showing the overall configuration of a molecular sequence analysis system applied to read the sequence of DNA molecule bases according to an embodiment of the present invention.

FIG. 2 is a cross-sectional view taken along line A-A of FIG. 1.

FIG. 3 is a perspective view showing various nanochannel shapes that may be used in the present invention.

FIG. 4 is a perspective view showing an example of the arrangement of a nanochannel and electrodes, which may be used in the present invention.

FIG. 5 is a perspective view showing an example of the arrangement of a nanochannel and electrodes, which may be used in the present invention.

FIG. 6 is a perspective view showing an example of the arrangement of electrodes in the case in which a nanochannel has no open side.

FIG. 7 is a perspective view and an enlarged perspective view, which illustrate an example of a measurement element connected to an electrode on a nanochannel by an extended gate.

FIG. 8 is a perspective view showing an example of the arrangement of a plurality of probe electrodes, which may be used in the present invention.

FIG. 9 is a perspective view showing an example of the arrangement of a plurality of probe electrodes, which may be used in the present invention.

FIG. 10 is a cross-sectional view taken along line B-B of FIG. 9.

FIG. 11 is a cross-sectional view taken along line C-C of FIG. 9.

FIG. 12 is a perspective view showing an example of the arrangement of probe electrodes coated with four different bases, which may be used in the present invention.

FIG. 13 is a perspective view showing an example of the arrangement of probe electrodes coated with four different bases, which may be used in the present invention.

FIG. 14 is a graphic diagram showing real-time measurement data that can be predicted using the four coated independent probe electrodes, which may be used in the present invention.

FIG. 15 is a perspective view showing an example of the arrangement of four probe electrode pairs applied with specific voltage values that allow resonant tunneling with the energy levels of different base molecules, which may be used in the present invention.

FIG. 16 is a graphic diagram showing real-time measurement data that can be predicted using the four probe electrode pairs applied with different specific voltages of FIG. 15, which may be used in the present invention.

### DESCRIPTION OF THE REFERENCE NUMERALS IN THE DRAWINGS

10: base sequence analysis system; 20: ss-DNA;

50: substrate; 100: nanochannel;

200: probe electrode; 200A: probe electrode coated with A;

200G: probe electrode coated with G;

200C: probe electrode coated with C;

200T: probe electrode coated with T;

210: single-layer electrode;

220: multilayer electrode; 222: lower layer electrode;

224: insulating layer; 225: upper layer electrode;

300: control electrode; 400: measurement element;

410: quantum dot; 411: source;

412: drain; 413: first gate;

414: second gate; 420: extended gate.

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated.

Hereinafter, preferred embodiments of a system 10 for analyzing the sequence of individual molecules according to the present invention will be described in detail with reference to the accompanying drawings.

In the following description of embodiments of the present invention, the detailed description of known elements obvious to those skilled in the art will be omitted so as not to obscure the gist of the present invention.

In the drawings, the thickness of lines or the size of constituent elements may be exaggerated for the clear understanding and convenience of description. Terms such as before, after, upper, lower, left, right, inner and outer, which indicate relative locations, are based on the direction shown in the drawings.

The system for analyzing the sequence of molecules using a nanochannel according to the present invention can be used to read the sequence of individual molecules of various biopolymers such as polypeptides, proteins or DNA (for example, the sequence of amino acid molecules of protein, the sequence of base molecules of DNA). In a specific embodiment, the system of the present invention may be used to analyze the sequence of base molecules of DNA as described below.

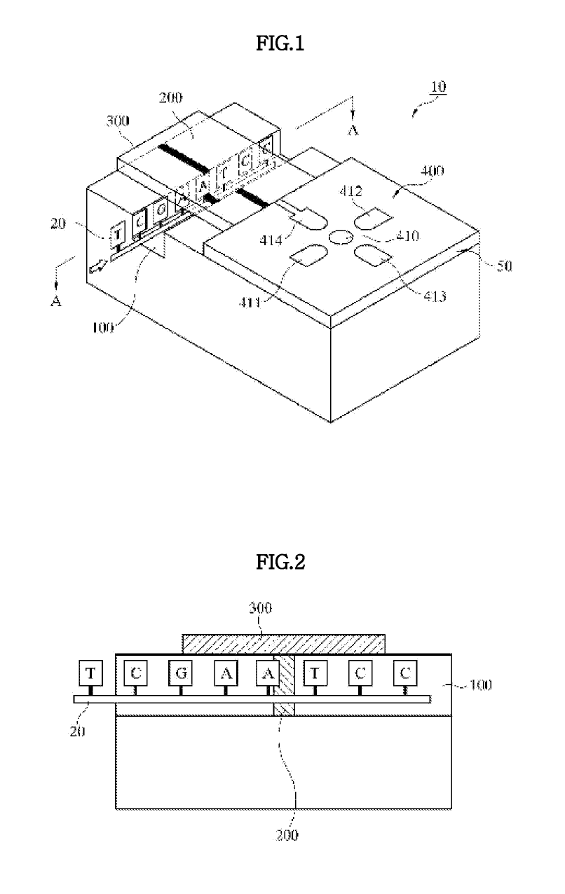

As shown in FIGS. 1 and 2, a molecular sequence analysis system 10 according to the present invention comprises a nanochannel 100, a probe electrode 200, a control electrode 300, and a measurement element 400.

The fundamental function of the inventive system having the above-described configuration is as follows. Either the change in charge distribution induced by the electric dipoles of different nucleotides of a single-stranded DNA (ss-DNA) 20 passing through the nanochannel or the change in current caused by a difference in orbital energy of the nucleotides is sensed by one or more probe electrodes 200, thereby analyzing the base sequence of the DNA. Separately from the probe electrode 200, the control electrode 300 is disposed on the nanochannel 100 in order to fix or align the bases of nucleotides in a uniform direction and control the moving speed of the bases, thereby increasing the accuracy and efficiency of analysis of the base sequence.

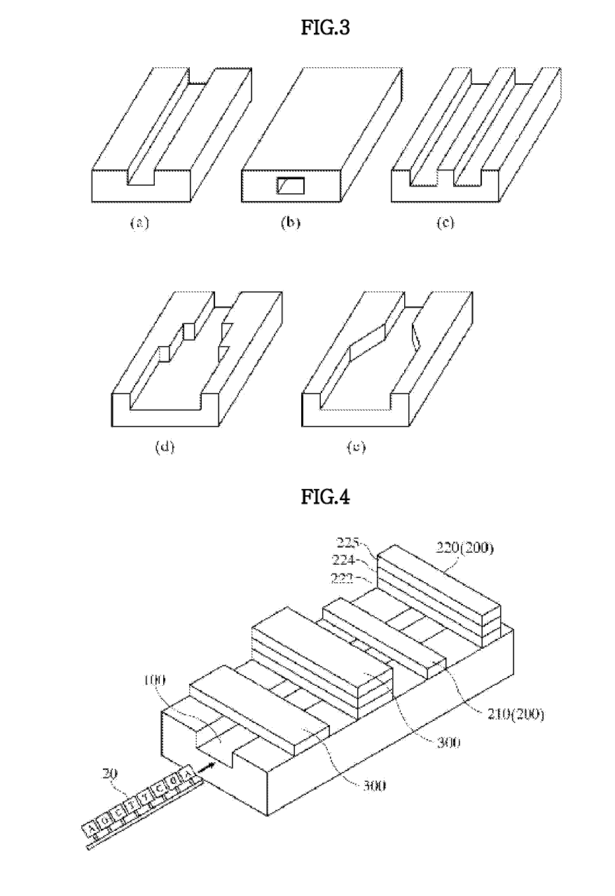

Hereinafter, each element of the system of the present invention will be described in detail. The nanochannel 100 has a width and height that allows the ss-DNA 20 to pass therethrough without twisting or folding. Generally, the width and height of the nanochannel 100 range from 0.1 nm to several hundred nm, and the ss-DNA 20 passes through the nanochannel 100 by electrophoresis or the difference in pressure of a fluid. FIG. 3 shows various examples that can be used as the nanochannel 100.

When the ss-DNA is used, the bases of the DNA are exposed so that either the change in potential induced by the electric dipoles of different nucleotides or the change in electric current caused by a difference in the orbital energy of the nucleotides can be sensed. Because one strand of double-stranded DNA (ds-DNA) has a sequence complementary to that of another strand, it is possible to analyze the base sequence of the ss-DNA 20.

The nanochannel 100 has a width and height that allows the ss-DNA 20 to pass therethrough without twisting or folding. As can be seen in FIGS. 3(d) and 3(e), the inlet of the nanochannel 100 may be made wider than other portions, and the width or height of the nanochannel 100 may be decreased continuously or stepwise toward the downstream side, and then made uniform so that the twisting or folding of the ss-DNA 20 does not occur. When the inlet of the nanochannel 100 is made wider than other portions, the initial introduction of the ss-DNA 20 will be easily induced. The probe electrode 200 (if necessary, including a control electrode) is preferably formed on a portion of the nanochannel, which has a width and height that allows the ss-DNA 20 to pass therethrough without twisting or folding.

Meanwhile, the control electrode 300 functions to align nucleotides in the same direction and control the moving speed of nucleotides during passage through the nanochannel. The control electrode 300 can be disposed on or under the nanochannel 100 or under a substrate 50 having the nanochannel 100 formed thereon in such a manner that it goes across the nanochannel 100. For example, FIGS. 4 and 5 show a control electrode 300 disposed on the open top of the nanochannel 100, in which the control electrode has a large width so that it can sufficiently interact with the ss-DNA 20 passing through the nanochannel 100. This control electrode 300 functions to align nucleotides, which are introduced into the nanochannel 100, in the same direction by the use of the electrical or chemical properties of the nucleotides, and control the moving speed of the nucleotides. In other words, the control electrode 300 functions to align the bases of the ss-DNA 20, which is introduced into the nanochannel 100, in a uniform direction, and thus fix the direction of the dipole moments, thereby increasing the sensing efficiency and accuracy of the probe electrode 200.

Herein, the use of the electrical properties of the nucleotides is the use of the negatively charged properties of phosphate groups present in the backbone of the ss-DNA 20. The phosphate groups of nucleotides have a negative charge. Thus, when a negative voltage is applied to the control electrode 300 disposed in the same plane as the probe electrode 200 or when the control electrode 300 is earthed, the phosphate groups will receive a repulsive force by the negative charge of the control electrode 300, and thus bases located opposite the phosphate groups (with respect to pentose located at the center) will be aligned to face the probe electrode 200. On the contrary, even when the control electrode 300 is disposed opposite the probe electrode 200 and a positive voltage is applied thereto, the same effect can be obtained. In addition, it is possible to float the control electrode 300.

The control electrode 300 as described above may be made of a conductive material including gold, silver, copper, platinum, palladium, titanium, nickel or cobalt, and may be composed of a single-layer electrode or a multilayer electrode, like the probe electrode 200.

Moreover, the use of the chemical properties of nucleotides to align is the use of the interaction (for example, p-p orbital interaction) between the bases of the nucleotides and graphene (or graphite or carbon nanotubes) that is the material of the control electrode. In other words, when the control electrode 300 is formed of a material, such as graphene, graphite or carbon nanotubes, which can interact with the bases of nucleotides, the direction of bases of nucleotides that pass below or above the control electrode 300 will be maintained uniformly by their interaction with the control electrode 300.

Furthermore, at least one probe electrode 200 is disposed adjacent to any one surface of the nanochannel 100 in a direction perpendicular to the lengthwise direction of the nanochannel 100. The probe electrode 200 functions to sense either the change in charge distribution induced by the dipole moment of different nucleotides of the ss-DNA 20 passing through the nanochannel 100, or the change in current caused by a difference in the orbital energy of the nucleotides. In other words, the probe electrode 200 refers to an electrode capable of sensing individual nucleotides.

Different nucleotides have different electric dipoles attributable to the respective charge distributions, and the change in charge distribution caused by the electric dipoles can be sensed by the probe electrode 200, thereby reading the type of nucleotide. As shown in FIGS. 1 and 2, the charge distribution of the probe electrode 200 is changed by the influence of the dipole moment produced by a base closest to the probe electrode 200 among a series of bases contained in the ss-DNA 20 passing through the nanochannel 100, and thus it is possible to read the type of base by sensing this change.

FIG. 4 shows a single-layer or multilayer probe electrode 200 disposed on the open top of the nanochannel 100. In this case, the ss-DNA 20 passing through the nanochannel 100 is aligned in a certain direction by the control electrode 300, and then the dipole moments of the base molecules are sensed by the probe electrode 200. On the other hand, FIG. 5 shows a probe electrode 200 disposed either at the vertical side of the nanochannel 100 or under the nanochannel 100. In this case, the ss-DNA 20 passing through the nanochannel 100 is aligned in a certain direction by a wide control electrode 300 formed on the channel, while the dipole moments of the bases are sensed by the probe electrode 200. This structure has an advantage in that, because all the probe electrodes 200 are disposed within the space covered by the control electrode 300, the direction of the nucleotides of the ss-DNA 20 passing through the channel is maintained uniformly by the control electrode while the dipole moments of the bases are sensed, thereby increasing the reliability of analysis.

The probe electrode 200 may be formed of a conductive or semi-conductive material including gold, silver, copper, platinum, palladium, titanium, nickel, cobalt, graphene, graphite, or and carbon nanotubes, which can transmit electrical signals.

In alternatives to the method of sensing the change in charge distribution induced by the electric dipole, it is also possible to analyze the base sequence of the ss-DNA 20 using the change in electric current caused by the orbital energy characteristics of nucleotides.

In a first alternative analysis method, a tunneling current in a direction perpendicular to the direction in which nucleotides pass through the nanochannel is measured using a probe electrode pair consisting of two opposite probe electrodes located at the two opposite sides of the nanochannel, respectively (FIG. 5). Because nucleotides have different energy levels, a change in the tunneling current flowing through the probe electrode pair is sensed by the measurement element 400, thereby determining the type of nucleotide base. In this case, the direction of the ss-DNA 20 passing through the nanochannel 100 can be aligned by the wide control electrode 300 formed on the nanochannel while the moving speed thereof can also be controlled.

In another analysis method, the probe electrode 200 (FIG. 4) disposed on the open top of the nanochannel is formed of a material capable of interacting with the orbital energy of nucleotide bases, and a change in an electric current flowing through the probe electrode 200 is measured, thereby determining the type of nucleotide. Because nucleotide bases have different orbital energies, resonance energy between nucleotide bases and the material of the probe electrode varies depending on the type of nucleotide base. Thus, the type of nucleotide base can be determined by measuring a minute change in the electric current passing through the probe electrode. If the probe electrode 200 is formed of a single-layer electrode 210, an electric current will flow from one end of the electrode to the other end, and if the probe electrode 200 is formed of a multilayer electrode 220, an electric current will flow from one end of a lower layer electrode 222 to the other end, and the Fermi energy of the lower layer electrode 222 will be controlled by controlling the voltage of an upper layer electrode 225. In this case, if energy resonance occurs between the orbital energy (e.g., p-orbital energy) of nucleotide bases and the material of the probe electrode material at a certain voltage, the interaction therebetween can be maximized, and a minute change in the electric current can be sensed.

However, the single-layer electrode 210 or the lower layer electrode 222 of the multilayer electrode 220 should be formed of a material capable of interacting with the orbital energy of nucleotide bases. For example, it should be formed of a material such as graphene, graphite, or carbon nanotubes. Between the lower layer electrode 222 and the upper layer electrode 225, an insulating layer 224 that insulates these electrodes from each other is formed. The probe electrode 200 may be composed of the single-layer electrode 210 or the multilayer electrode 220, and at least a portion of the top of the single-layer electrode 210 or the upper and lower layers of the multilayer layer electrode 220 may be coated with a thin dielectric layer. The dielectric layer is formed for the purposes of providing electrical insulation and increasing the sensitivity of measurement.

For the same purposes, at least a portion of the inner surface of the nanochannel 100 may also be coated with a dielectric layer. Particularly, it is effective to form a dielectric layer at the boundary between the probe electrode 200 and the nanochannel 100.

It is to be understood that the above-described configuration of the single-layer electrode, the multilayer electrode or the dielectric layer may, if necessary, be modified in various ways.

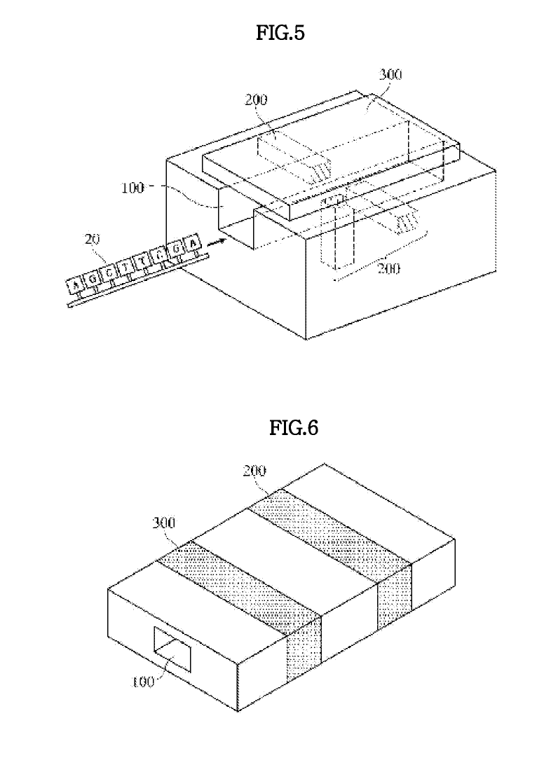

Meanwhile, as shown in FIGS. 1 to 4, one side of the nanochannel 100 may be open, and at least one of the probe electrode 200 and the control electrode 300 may be disposed on the open side of the nanochannel 100. However, as shown in FIG. 6, all the sides of the nanochannel 100, excluding the inlet and the outlet, may be closed (FIG. 3(b)). In this case, the probe electrode 200 and the control electrode 300 may be formed on any one side of the nanochannel 100, like the case in which any one side of the nanochannel is open. Alternatively, the probe electrode 200 may also be formed in a direction perpendicular to the lengthwise direction of the nanochannel 100. This is because it is advantageous in terms of accuracy and speed to sense and measure the nucleotide sequence of the ss-DNA 20, which passes through the nanochannel 100, at a closer position.

With respect to the measurement element 400, either the change in potential induced by the electric dipoles of nucleotides, or the change in current caused by a difference in orbital energy, is sensed by the probe electrode, and the absolute value or relative value of this change is measured by the measurement element 400 electrically connected to the probe electrode 200. In other words, the measurement element 400 can measure the changes in charge distribution and current, which change depending on the type of nucleotide, thereby determining the type of nucleotide.

The measurement element 400 that is used in the present invention may be composed of a field effect transistor (FET), an operational amplifier, a single electron transistor (SET) or a quantum point contact (QPC). FIGS. 1 and 6 show the overall configuration of a single electron transistor comprising: a quantum dot 410 having a size ranging from several nm to several tens of nm; a source 411 configured to emit electrons; a drain configured to receive electrons from the quantum dot 410; a first gate 413 configured to control the state of the quantum dot 410; and a second gate 414 required to couple the probe electrode 200 to the quantum dot 410.

Also, in order to further increase the measurement speed and sensitivity of the measurement element 400, a high-frequency (RF) resonance circuit may be attached to any one or both of the source 411 or drain 412 of the measurement element 400, so that high frequency can be applied to thereby measure a change in high-frequency transmission or reflection. In addition, an additional amplifier may be disposed at a position closest to the source 411 or drain 412 of the measurement element 400, so that the signal of the additional amplifier can be sensed. The measurement element 400 that employs high frequency may be, for example, a high-frequency single-transistor transistor (RF-SET) or a high-frequency quantum dot contact (RF-QPC).

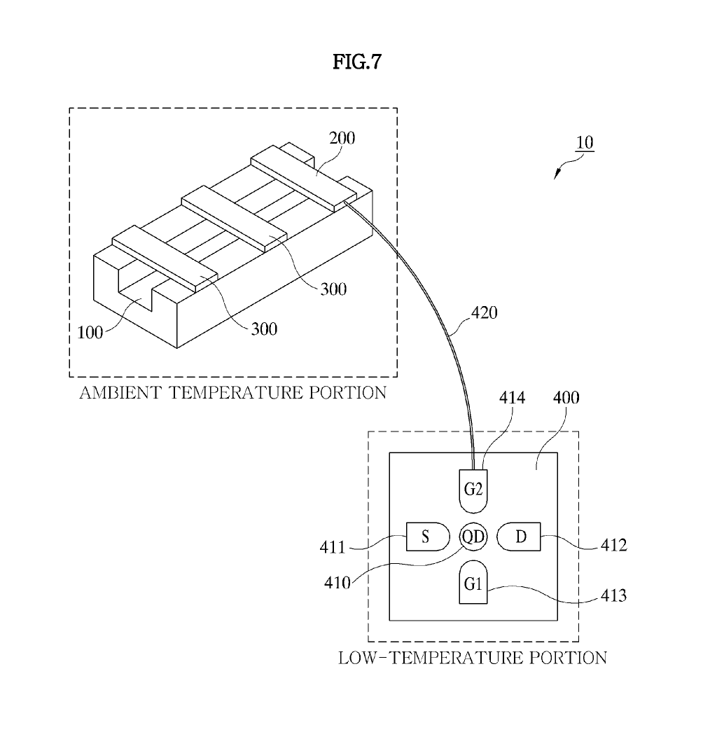

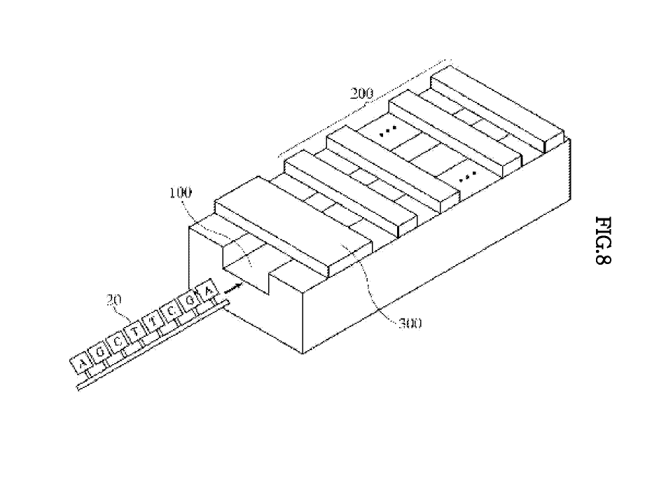

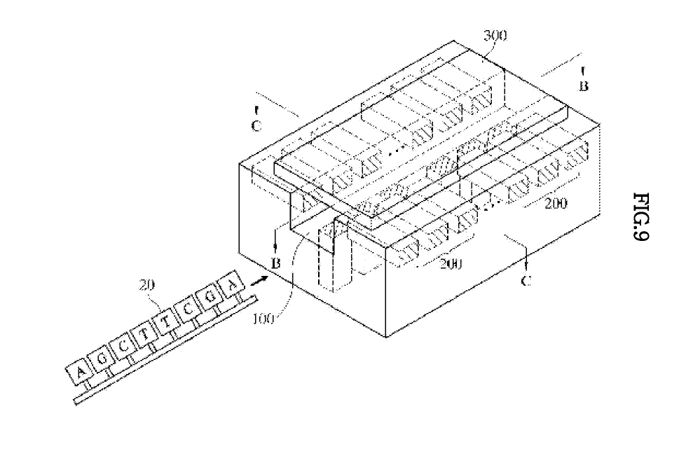

In addition, the measurement element 400 may be configured such that it is electrically connected to the probe electrode 200 through an extended gate 420 and is at an atmosphere temperature lower than the temperature surrounding the nanochannel 100. This embodiment is illustrated in FIG. 7. In this embodiment, the temperature surrounding the measurement element 400 can be lowered to reduce the intrinsic noise of the measurement element 400, thereby more clearly sensing the signal of the probe electrode 200.

In addition, the sequence analysis system 10 according to the present invention may be configured to have a simplified structure by forming the nanochannel 100 on the substrate 50 and forming the measurement element 400 on the substrate 50 (see FIG. 1). Particularly, when the system is simplified by forming the measurement element 400, which is sensitive to charges, directly on the substrate 50 having the nanochannel 100 formed thereon, and then connecting the measurement element 400 to the electrode, the effects of increasing the measurement speed and reducing the extrinsic noise can be obtained.

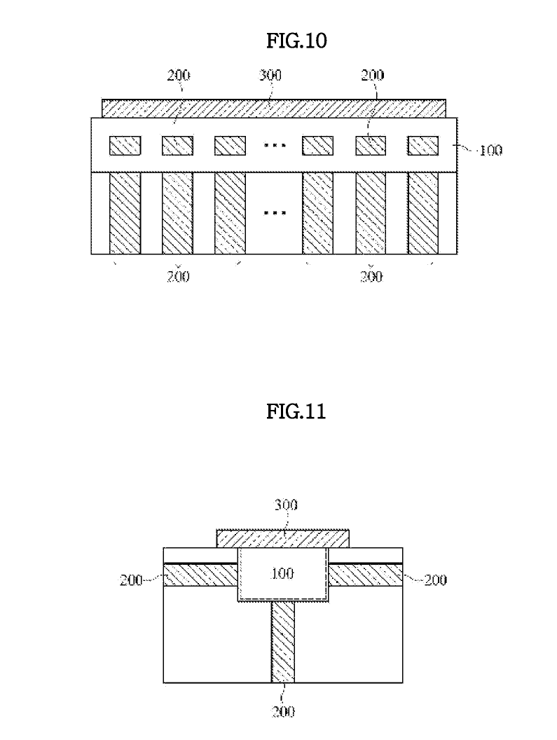

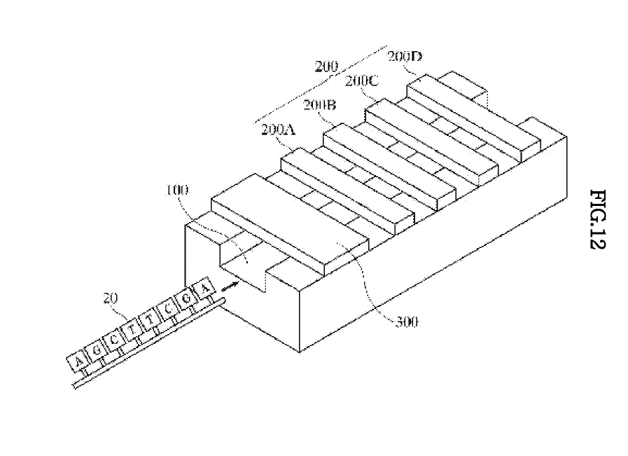

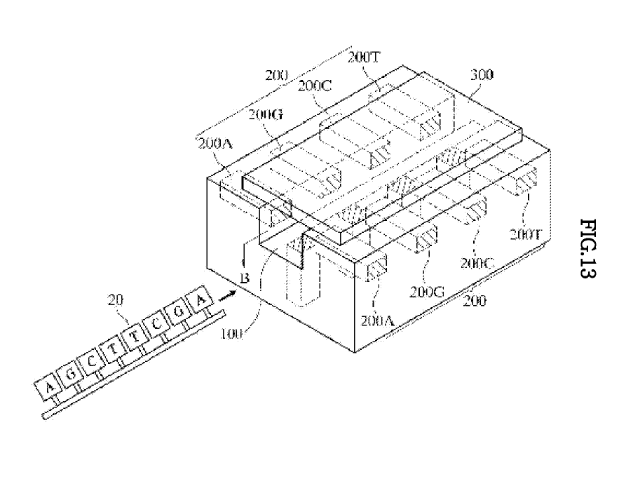

Meanwhile, each of the nanochannel 100, the probe electrode 200, the control electrode 300 and the measurement element 400, which are included in the sequencing system 10 of the present invention, may be one or more in number. For example, FIG. 8 illustrates that a plurality of probe electrodes 200 are formed following the control electrode 300 on the open top of the nanochannel 100 in a row along the lengthwise direction of the nanochannel 100. On the other hand, FIG. 9 illustrates that the control electrode is formed on the nanochannel 100 and a plurality of probe electrodes 200 are formed either at the vertical sides of the nanochannel 100 or under the nanochannel so as to be arranged in a row along the lengthwise direction of the nanochannel 100.

The structures having the plurality of probe electrodes (FIGS. 8 and 9) may be applied to the above-described methods of analyzing molecular sequences using nanochannels (that is, analytic methods that sense either the change in charge distribution induced by the electric dipoles of different nucleotides or the change in current caused by a difference in the orbital energy of nucleotides). Such structures have advantages in that, because a plurality of probe electrode sets having the same configuration are formed per nanochannel, all nucleotides passing through the nanochannel during passage of one ss-DNA can be individually read a plurality of times, and thus the reliability of analysis can be increased while the time required for analysis can be greatly reduced. This is the most important key element in the implementation of the present invention, and it is to be understood that, as the number of the plurality of probe electrodes arranged in a row increases, the speed and reliability of nucleotide analysis increase. However, all the probe electrodes should be disposed within the range of the length of the nanochannel.

In addition, in a method that is applied to the methods for analyzing the sequence of molecules using a nanochannel, the probe electrodes may be coated with complementary molecules capable of chemically bonding to ss-DNA base molecules passing through the nanochannel, in order to enhance their interactions with the ss-DNA base molecules. This method can be applied to the case in which either the change in charge distribution induced by the electric dipoles of different nucleotides of a ss-DNA or the change in current caused by a difference in the orbital energy of nucleotides is insignificant so that noise cannot be overcome by the probe electrode. For example, as shown in FIGS. 12 and 13, four independent probe electrodes 200 are formed per nanochannel, and are coated with four different types of DNA base molecules or deoxyribonucleotides, respectively, so that they can form a complementary chemical bond (T-A or C-G) with the ss-DNA (base sequence: AGCTTCGA) passing through the nanochannel, thereby maximizing the sensing efficiency.

Among the probe electrodes 200 shown in FIGS. 12 and 13, the probe electrode 200A is a probe electrode coated with either adenine or a deoxyribonucleotide (dATP) having adenine as a base; the probe electrode 200G is a probe electrode coated with either guanine or a deoxyribonucleotide (dGTP) having guanine as a base; the probe electrode 200C is a probe electrode coated with either cytosine or a deoxyribonucleotide (dCTP) having cytosine as a base; and the probe electrode 200T is a probe electrode coated with either thymine or a deoxyribonucleotide (dTTP) having thymine as a base.

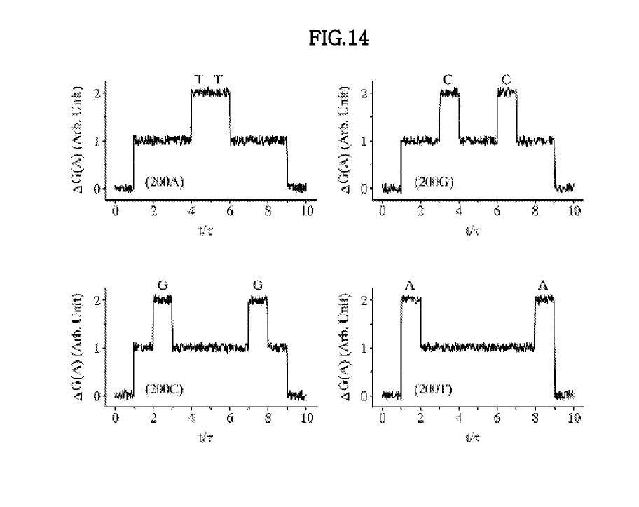

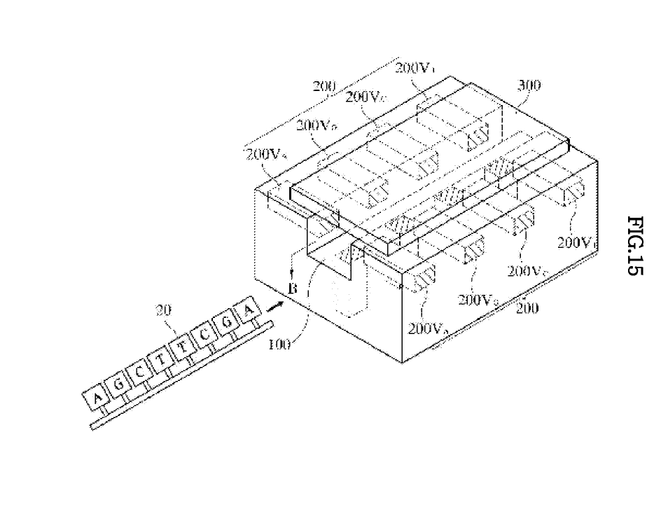

FIG. 14 shows real-time measurement data that can be predicted for the ss-DNA (base sequence: AGCTTCGA) passing through the nanochannel by the use of the four independent probe electrodes coated with different bases. Taking these four real-time data together, the base sequence (AGCTTCGA) of the ss-DNA that passed through the nanochannel can be read.

In another method that is applied to the method of analyzing the sequence of base molecules using a nanochannel and a plurality of probe electrode pairs, a specific voltage is applied through the pairs of opposite probe electrodes so as to allow resonant tunneling between the probe electrode pair and any one base molecule among the four different base molecules of the ss-DNA passing through the nanochannel. For example, as shown in FIG. 15, at least four independent probe electrode pairs 200 are formed around the nanochannel, and a specific voltage obtained by controlling the Fermi energy is applied to and maintained in any one of the probe electrode pairs so as to allow resonant tunneling between the probe electrode pair and any one of the four types of DNA base molecules or deoxyribonucleotides.

Among the four probe electrode pairs 200 shown in FIG. 15, 200V_{A} means a specific voltage applied so as to allow resonant tunneling between the probe electrode pair and the molecular orbit of adenine or a deoxyribonucleotide (dATP) having adenine as a base; 200V_{G} means a specific voltage applied pair so as to allow resonant tunneling between the probe electrode pair and the molecular orbit of guanine or a deoxyribonucleotide (dGTP) having guanine as a base; 200V_{C} means a specific voltage applied so as to allow resonant tunneling between the probe electrode pair and the molecular orbit of cytosine or a deoxyribonucleotide (dCTP) having cytosine a base; and 200V_{T} means a specific voltage applied so as to allow resonant tunneling between the probe electrode pair and the molecular orbit of thymine or a deoxyribonucleotide (dCTP) having thymine a base.

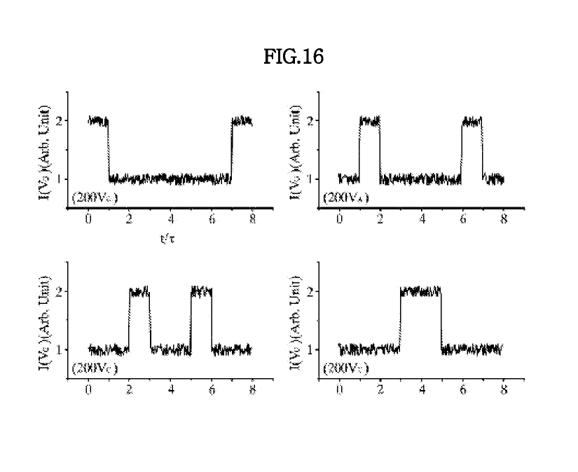

FIG. 16 shows real-time measurement data that can be predicted using the four independent probe electrode pairs applied with different specific resonant tunneling voltages for the ss-DNA (base sequence: GACTTCAG) passing through the nanochannel, as described above with respect to FIG. 15. Taking these four real-time data together, the base sequence (GACTTCAG) of the ss-DNA that passed through the nanochannel can be read.

Although the preferred embodiments of the present invention have been disclosed for illustrative purposes with reference to the accompanying drawings, those skilled in the art will appreciate that various modifications, additions and substitutions are possible. For example, a plurality of nanochannels may be formed in parallel on a single substrate, and various parts of a single ss-DNA may be passed through the nanochannels and analyzed individually at the same time using the base sequence analysis system of the present invention. In this case, the time required to read the sequence of all the individual molecules of the ss-DNA.

This completes the description of the preferred and alternate embodiments of the invention. Those skilled in the art may recognize other equivalents to the specific embodiment described herein which equivalents are intended to be encompassed by the claims attached hereto.

What is claimed is:

1. A system for analyzing a sequence of molecules using a nanochannel, the system comprising:
at least one nanochannel having a width and height that allows a biopolymer to pass therethrough without twisting or folding;
at least one control electrode disposed on any one side of the nanochannel across the nanochannel and configured to align individual molecules of the biopolymer, which is introduced into the nanochannel, in the same direction according to electrical or chemical properties of the individual molecules, and to control moving speed of the individual molecules;
at least one probe electrode, one end or side of which is disposed adjacent to any one side of the nanochannel along a direction perpendicular to a lengthwise direction of the nanochannel and which is configured to sense either a change in charge distribution induced by electric dipoles of different individual molecules of the biopolymer passing through the nanochannel or a change in current caused by a difference in orbital energy of the individual molecules; and
a measurement element configured to measure an absolute or relative value of the change in charge distribution or current sensed by each of the probe electrodes.

2. The system of claim 1, wherein any one of the width and height of the nanochannel decreases continuously or stepwise from an inlet toward a downstream side thereof, and is then uniform so that the biopolymer passes through the nanochannel without twisting or folding.

3. The system of claim 1, wherein at least a portion of an inner surface of the nanochannel is coated with a dielectric layer.

4. The system of claim 1, wherein the control electrode is made of a conductive material including gold, silver, copper, platinum, palladium, titanium, nickel or cobalt, and is disposed over or under the nanochannel or under a substrate so that it is applied with a specific voltage, earthed or floated.

5. The system of claim 1, wherein the control electrode is made of a material including any one of graphene, graphite, and carbon nanotubes, which is capable of interacting with the individual molecules of the biopolymer, and the control electrode is disposed over or under the nanochannel or under a substrate so that it is applied with a specific voltage, earthed or floated.

6. The system of claim 1, wherein the probe electrode is formed of a conductive or semi-conductive material including any one of gold, silver, copper, platinum, palladium, titanium, nickel, cobalt, graphene, graphite, and carbon nanotubes.

7. The system of claim 1, wherein each of the probe electrode and the control electrode is composed of a single-layer electrode or a multilayer electrode, and at least a portion of a lower portion of the single-layer electrode or the upper and lower layers of the multilayer electrode is coated with a dielectric layer.

8. The system of claim 1, wherein the measurement element is any one of a field-effect transistor (FET), an operational amplifier, a single-electron transistor (SET), a high-frequency single-electron transistor (RF-SET), a quantum point contact (QPC) and a high-frequency quantum point contact (RF-QPC).

9. The system of claim 1, wherein the measurement element is electrically connected with the probe electrode through an extended gate and is at an atmosphere temperature lower than an atmosphere temperature of the nanochannel.

10. The system of claim 1, wherein the measurement element is formed integrally on a substrate having the nanochannel formed thereon.

11. The system of claim 1, wherein a plurality of the probe electrodes are formed following the control electrode over an open top of the nanochannel in a row along a lengthwise direction of the nanochannel, and the probe electrodes are connected to different measurement elements.

12. The system of claim 1, wherein the control electrode is formed over an open top of the nanochannel, and a plurality of the probe electrodes are arranged at vertical sides of the nanochannel or under the nanochannel a lengthwise direction of the nanochannel, and the probe electrodes are connected to different measurement elements.

13. The system of claim 1, wherein the control electrode is formed over an open top of the nanochannel, and a plurality of probe electrode pairs, each consisting of two opposite probe electrodes located at two opposite sides of the nanochannel, respectively, are disposed, and the plurality of probe electrode pairs are connected to different measurement elements.

14. The system of claim 11, wherein at least four probe electrodes are formed within a range of the length of the nanochannel, and the probe electrodes are coated with complementary molecules capable of chemically bonding with the individual molecules passing through the channel, respectively.

15. A method for analyzing a sequence of molecules using a nanochannel, the method comprising the steps of:
moving a biopolymer in a nanochannel by electrophoresis or a difference in pressure of a fluid;
applying a voltage to a control electrode formed over or under the nanochannel or under a substrate having the nanochannel formed thereon, or connecting the control electrode to an earth, or floating the control electrode, to align individual molecules of the biopolymer in a uniform direction and control a moving speed of the individual molecules;
inducing a change in charge distribution of a probe electrode by electric dipoles of the individual molecules of the biopolymer; and
transferring the change in charge distribution of the probe electrode to a measurement element to read the type of individual molecules.

16. A method for analyzing a sequence of molecules using a nanochannel, the method comprising the steps of:
moving a biopolymer in a nanochannel by electrophoresis or a difference in pressure of a fluid;
applying a voltage to a control electrode formed over or under the nanochannel or under a substrate having the nanochannel formed thereon, or connecting the control electrode to an earth, or floating the control electrode, to align individual molecules of the biopolymer in a uniform direction and control a moving speed of the individual molecules;
tunneling energy levels of the individual molecules through a probe electrode pair consisting of two opposite probe electrodes located at two opposite sides of the nanochannel, respectively; and
sensing a change in the tunneling currents by a measurement element connected to the probe electrode pair to read the type of individual molecules.

17. A method for analyzing a sequence of molecules using a nanochannel, the method comprising the steps of:
moving a biopolymer in a nanochannel by electrophoresis or a difference in pressure of a fluid;
applying a voltage to a control electrode formed over or under the nanochannel or under a substrate having the nanochannel formed thereon, or connecting the control electrode to an earth, or floating the control electrode, to align individual molecules of the biopolymer in a uniform direction and control a moving speed of the individual molecules;
interacting the individual molecules with a single-layer probe electrode or a lower layer electrode of a multilayer probe electrode, disposed over an open top of the nanochannel; and
sensing either a change in current of the single-layer probe electrode or a change in current of the lower layer electrode of the multilayer probe electrode, caused by a change in a voltage applied to the upper layer electrode, by a measurement element connected to the single-layer probe electrode or the lower layer electrode of the multilayer probe electrode, to read the types of individual molecules.

18. The method of claim 15, wherein a plurality of probe electrodes or probe electrode pairs having the same configuration are formed within a range of the length of the nanochannel, so that the individual molecules of the biopolymer passing through the nanochannel are individually read a plurality of times, thereby increasing reliability of the analysis while reducing the time required for the analysis.

19. The method of claim 15, wherein at least four probe electrodes or probe electrode pairs having the same configuration are formed within a range of the length of the nanochannel, and coated with complementary molecules capable of chemically bonding with the individual molecules, respectively, in order to enhance their interaction with the individual molecules, thereby maximizing sensing efficiency.

20. The method of claim 15, wherein at least four probe electrode pairs having the same configuration are formed within a range of the length of the nanochannel, and the probe pair electrodes are applied with four different specific voltages, respectively, so that resonant tunneling with energy levels of four types of base molecules passing through the nanochannel occurs.

21. The system of claim 12, wherein at least four probe electrodes are formed within a range of the length of the nanochannel, and the probe electrodes are coated with complementary molecules capable of chemically bonding with the individual molecules passing through the channel, respectively.

22. The system of claim 13, wherein at least four probe electrodes are formed within a range of the length of the nanochannel, and the probe electrodes are coated with complementary molecules capable of chemically bonding with the individual molecules passing through the channel, respectively.

23. The method of claim 16, wherein a plurality of probe electrodes or probe electrode pairs having the same configuration are formed within a range of the length of the nanochannel, so that the individual molecules of the biopolymer passing through the nanochannel are individually read a plurality of times, thereby increasing reliability of the analysis while reducing the time required for the analysis.

24. The method of claim 17, wherein a plurality of probe electrodes or probe electrode pairs having the same configuration are formed within a range of the length of the nanochannel, so that the individual molecules of the biopolymer passing through the nanochannel are individually read a plurality of times, thereby increasing reliability of the analysis while reducing the time required for the analysis.

25. The method of claim 16, wherein at least four probe electrodes or probe electrode pairs having the same configuration are formed within a range of the length of the nanochannel, and coated with complementary molecules capable of chemically bonding with the individual molecules, respectively, in order to enhance their interaction with the individual molecules, thereby maximizing sensing efficiency.

26. The method of claim 17, wherein at least four probe electrodes or probe electrode pairs having the same configuration are formed within a range of the length of the nanochannel, and coated with complementary molecules capable of chemically bonding with the individual molecules, respectively, in order to enhance their interaction with the individual molecules, thereby maximizing sensing efficiency.

27. The method of claim 16, wherein at least four probe electrode pairs having the same configuration are formed within a range of the length of the nanochannel, and the probe pair electrodes are applied with four different specific voltages, respectively, so that resonant tunneling with energy levels of four types of base molecules passing through the nanochannel occurs.

28. The method of claim 17, wherein at least four probe electrode pairs having the same configuration are formed within a range of the length of the nanochannel, and the probe pair electrodes are applied with four different specific voltages, respectively, so that resonant tunneling with energy levels of four types of base molecules passing through the nanochannel occurs.
